(19) United States
(12) Patent Application Publication
(10) Pub. No.: US 2020/0362331 A1
(43) Pub. Date: Nov. 19, 2020

(54) Small Enzyme Particles For Interesterification

(71) Applicant: Novozymes A/S, Bagsvaerd (DK)

(72) Inventors: Per Munk Nielsen, Hilleroed (DK); Hans Christian Holm, Hellerup (DK); Pavle Andric, Copenhagen (DK)

(73) Assignee: Novozymes A/S, Bagsvaerd (DK)

(21) Appl. No.: 16/766,189

(22) PCT Filed: Nov. 23, 2018
(86) PCT No.: PCT/EP2018/082419

§ 371 Date: May 21, 2020

(30) Foreign Application Priority Data
Nov. 24, 2017 (EP) 17203622.0

(51) Int. Cl. C12N 11/14; C12N 11/12; C12P 7/64

(52) U.S. Cl. CPC C12N 11/14; C12P 7/6454; C12N 11/12

(57) ABSTRACT

The invention provides enzyme particles comprising an immobilized lipolytic enzyme, a siliceous material, an organic filter aid, and a water-soluble polyol selected from carbohydrates and sugar alcohols. The particles are suitable for enzymatic interesterification of triglycerides, and subsequent separation of the enzyme and triglycerides by filtration.

### FIELD OF THE INVENTION

The present invention relates to small lipolytic enzyme particles for use in an enzymatic interesterification process, which have advantageous properties in both reaction rate and subsequent separation processes.

### BACKGROUND

Immobilization of lipolytic enzymes has been known for many years. An immobilized enzyme product may be used in enzymatic modification of an organic compound such as in organic synthesis processes, vegetable oil interesterification, biodiesel production etc.

Enzyme immobilization is the attachment of an enzyme protein on a carrier on which the enzyme is fixed, yet functional, where the enzyme is not released into the liquid (washed out) to which it is contacted. The most commonly immobilized enzymes are glucose isomerase used for isomerization reactions, and lipase used for, e.g., interesterification of vegetable oils and organic synthesis.

The industrial use of enzymes is often limited by their high cost and rapid inactivation. To improve their economic feasibility in industrial processes, enzymes are generally immobilized onto a particle. Immobilization facilitates re-use of the enzymes, and may affect the selectivity and stability of the enzyme. Immobilization research has mainly focused upon means to enhance the transfer of enzymes onto the support, and means to ensure that the enzymes remain active after being immobilized.

For use in non-aqueous solutions, lipolytic enzymes, such as lipases, can be immobilized on a number of different porous, inorganic carriers by absorption of an aqueous solution of lipase into the pore volume of the carrier, or by adsorption to the surface of the carrier, or by a combination of both adsorption and absorption followed by water removal by drying.

JP 5-292965A discloses an immobilized lipase and a method for preparing it.

WO 95/22606 (Pedersen et al.) describes an immobilization process based on a granulation process.

WO 99/33964 (Christensen et al.) describes an immobilization process wherein the enzyme is applied to a particulate porous carrier.

Immobilized enzymes are known to be used in both continuous and batch enzymatic reactions within a variety of industrial applications, including waste water treatment, production of pharmaceuticals, high fructose corn syrup production, vegetable oil processing and synthesis of chemicals.

### SUMMARY OF THE INVENTION

In a first aspect, the present invention provides (a plurality of) enzyme particles, comprising a lipolytic enzyme, a siliceous material, an organic filter aid, and a water-soluble polyol selected from carbohydrates and sugar alcohols.

In another aspect of the invention is provided a method for enzymatic interesterification, comprising contacting a mixture of triglycerides with the enzyme particles of the invention.

Other aspects and embodiments of the invention are apparent from the description and examples.

### DETAILED DESCRIPTION

The use of (lipolytic) enzyme catalyzed interesterification of fats/oils is well established. The reaction takes place in columns with a height of 1-5 meters filled with immobilized enzyme with a typical particle size of 300-1200 μm, where the oil is pumped through a set of column with the total holding time required to achieve a certain conversion.

This type of setup (fixed bed column) is dependent upon an appropriate particle size of the enzyme to limit the pressure drop in the column. Another limitation for this kind of process is the mass transfer of oil into the particle. The easiest part of the enzyme activity to get access to is located at the surface, or in the near proximity to the particle surface. Therefore, the particle size for the process has until now been a compromise between having a size not too small to prevent high pressure drop, but still as small as possible to get a large surface area per kg of enzyme product. When using a large and robust particle size, it is necessary to take into account how much oil is held in the space between particles (voidage) as this oil will be mixed into the next batch of oil processed. This amount of oil can be so large that it in practice limits the use of this technology to productions with large volumes of same recipe to prevent mixing with oil from a previous batch.

With this invention, we have found a way to operate interesterification of oils/fats with a very small particle size (very high surface area), which results in an exceptionally high enzymatic activity. As explained above, this means that a lower amount of enzyme is needed because it is more accessible, and further that a lower amount of oil is trapped between the particles.

By the invented formulation of the particle it has been secured that the immobilized enzyme can be used either in batch/tank operation and filtered off after the reaction in standard oil filters, or it can be used in a fixed bed operation with a thin layer of enzyme such as 2-5 cm. The low dosage, low oil entrapment and possibility of re-using the enzyme provides a significantly improvement in cost-in-use compared to present technology.

Unless otherwise indicated, all percentages are indicated as percent by weight (')/0 w/w) throughout the application.

### Enzyme Particles

The particles of the invention comprise an immobilized lipolytic enzyme, a siliceous material, an organic filter aid, and a water-soluble polyol selected from carbohydrates and sugar alcohols. The particles may be encapsulated in oil or fat; e.g. to form an oily powder or a slurry/suspension.

The particles may be a homogenous mixture of the ingredients, i.e. the ingredients are uniformly distributed throughout the particles. Even if the individual particle is not uniform on a microscopic level, the ingredients may be randomly distributed with no overall structure, when a plurality of particles, such as at least 50 particles, is considered.

The particles are preferably porous. The pore volume may correspond to an oil uptake of at least 0.5 gram of oil per gram of particles, particularly at least 1 gram of oil per gram of particles. It may have a surface area of 5-1000 m²/g, 10-1000 m²/g, in particular 10-700 m²/g, more particularly 10-500 m²/g.

The particles may have a volume-based particle size (D₅₀) below 100 μm, preferably 1-60 μm, more preferably 2-40 μm, and particularly 5-30 μm. The particle size is measured with a laser diffraction particle size analyzer.

The particles may comprise the siliceous material and the organic filter aid in a total amount of 40-95% w/w, preferably 50-90% w/w.

In addition to the claimed ingredients, the particles may comprise inorganic, organic or both inorganic and organic material(s), which may be essentially insoluble in hydrophilic or hydrophobic liquids or mixtures thereof. The particles may further have a hydrophilic or hydrophobic surface. The particle surface can be modified and the enzyme may further be linked by hydrogen, ionic or covalent bonds or covalently cross-linked by, for example, glutaraldehyde treatment.

The particles may be prepared by spray drying a liquid (aqueous) mixture of the ingredients making up the particles (a siliceous material, an organic filter aid, enzyme and a water-soluble polyol selected from carbohydrates and sugar alcohols) or by absorption of the liquid solution of enzyme and a water-soluble polyol selected from carbohydrates and sugar alcohols, (separately or in a mixture) into a mixture of organic filter aid and siliceous materials followed by suitable drying technique (drying in a fluid bed, vacuum drier, etc.). The whole process could also be carried out in a combined/integrated mixer and dryer, such as a vacuum mixer. The mixture of filter aid and siliceous material (and any additional ingredients) may be preformed particles. By “preformed particles” is meant particles having their final form and structure before adding the enzyme and polyol. Generally, the ingredients can be added simultaneously or sequentially to optimize the production process.

The particles may contain less than 40% w/w water, preferably less than 25% w/w water, more preferably less than 10% w/w water, and most preferably less than 5% w/w water.

In order to make a final product with low dust properties and/or improved compatibility with the process in which it will be used (e.g., an interesterification process), the resulting particles can be sprayed with oil, or be blended with oil to obtain an oily powder or a slurry/suspension that encapsulates the particles in oil. The oil may be a plant derived oil, such as sunflower oil or another oil which is compatible with the process in which the particles will be used. If only a small amount of oil is used, the particles can be agglomerated into larger particles that can substantially reduce the amount of dust. The oil-encapsulated particles can also be dried subsequently.

The particles can also be sprayed or blended with fat to produce a solid block containing fat and small particles, or processed through extrusion and pelletizing equipment to obtain large pellets, which may also include added fat as a ‘vehicle’. Such particles can subsequently be coated with a preservation agent, for example a powderized preservation agent.

Dust is defined as particles with an aerodynamic diameter less than 50 μm. In aerosol science, it is generally accepted that particles with an aerodynamic diameter higher than 50 μm do not commonly remain airborne for very long. In this context, the aerodynamic diameter is defined as “the diameter of a hypothetical sphere of density 1 g/cm³ having the same terminal settling velocity in calm air as the particle in question, regardless of its geometric size, shape and true density.” (WHO, 1997).

### Lipolytic Enzyme

The enzyme to be immobilized according to the invention is a lipolytic enzyme, i.e. an enzyme which is capable of hydrolyzing carboxylic ester bonds to release carboxylate (EC 3.1.1). The lipolytic enzyme is an enzyme classified under the Enzyme Classification number E.C. 3.1.1.-(Carboxylic Ester Hydrolases) in accordance with the Recommendations (1992) of the International Union of Biochemistry and Molecular Biology (IUBMB). Thus, the lipolytic enzyme may exhibit hydrolytic activity, typically at a water/lipid interface, towards carboxylic ester bonds in substrates such as mono-, di- and triglycerides, phospholipids, thioesters, cholesterol esters, wax-esters, cutin, suberin, synthetic esters or other lipids mentioned in the context of E.C. 3.1.1. The lipolytic enzyme may, e.g., have triacylglycerol lipase activity (EC 3.1.1.3; 1,3-positionally specific or non-specific), phospholipase activity (A1 or A2; EC 3.1.1.32 or EC 3.1.1.4), esterase activity (EC 3.1.1.1) or cutinase activity (EC 3.1.1.74).

Suitable lipolytic enzymes (e.g. lipases) include those of bacterial or fungal origin. Chemically modified or protein engineered mutants are included. Examples include lipases from Candida, C. Antarctica (e.g. lipases A and B described in WO 88/02775), C. rugosa (C. cylindracea), Rhizomucor, R. miehei, Hyphozyma, Humicola, Thermomyces, T. lanuginosus (H. lanuginosa lipase) as described in EP 258 068 and EP 305 216, a Pseudomonas lipase, e.g. from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422), lipase/phospholipase from Fusarium oxysporum, lipase from F. heterosporum, lysophospholipase from Aspergillus foetidus, phospholipase A1 from A. oryzae, lipase from A. oryzae, lipase/ferulic acid esterase from A. niger, lipase/ferulic acid esterase from A. tubingensis, lipase from A. tubingensis, lysophospholipase from A. niger and lipase from F. solani.

The lipase may be positionally site specific (i.e., 1,3 specific) or non-specific, upon interaction with triglycerides as substrates.

Furthermore, a number of cloned lipases may be useful, including the Penicillium camembertii lipase described by Yamaguchi et al., (1991), Gene 103, 61-67), the Geotricum candidum lipase (Shimada, Y. et al., (1989), J. Biochem., 106, 383-388), and various Rhizopus lipases such as a R. delemar lipase (Hass, M. J et al., (1991), Gene 109, 117-113), a R. niveus lipase (Kugimiya et al., (1992), Biosci. Biotech. Biochem. 56, 716-719) and a R. oryzae lipase.

Other types of lipolytic enzymes such as cutinases may also be useful, e.g. cutinase from Pseudomonas mendocina (WO 88/09367), Fusarium solani pisi (WO 90/09446) or H. insolens (U.S. Pat. No. 5,827,719).

The enzyme may be an enzyme variant produced, for example, by recombinant techniques. Examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Examples of commercially available lipases include Lipex™, Lipoprime™, Lipolase™, Lipolase™ Ultra, Lipozyme™, Palatase™, Novozym™ 435, Quara™ and Lecitase™ (all available from Novozymes A/S). Other commercially available lipases include Lumafast™ (Pseudomonas mendocina lipase from Genencor International Inc.); Lipomax™ (Ps. pseudoalcaligenes lipase from DSM/Genencor Int. Inc.; and Bacillus sp. lipase from Genencor enzymes. Further lipases are available from other suppliers.

The enzyme may be added to the immobilization process in liquid form, such as an enzyme containing liquid (aqueous) medium.

The enzyme containing liquid medium is, in a particular embodiment of the present invention, a hydrophilic medium. In another particular embodiment, the liquid medium is aqueous. It may contain other organic or biological matter. Thus, it may be a fermentation broth or an enzyme concentrate solution obtainable by purifying a fermentation broth by e.g. ultra filtration or by protein precipitation, separation and re-dissolution in another aqueous medium. It may further be substantially pure enzyme dissolved in an aqueous medium. In a special embodiment of the present invention the enzyme containing aqueous liquid has not been subjected to costly processing steps prior to immobilization to remove water such as evaporation nor has it been subjected to addition of non-aqueous solvents, e.g. organic solvents such as alcohols, e.g. (poly)ethylene glycol and/or (poly) propylene glycol.

In one embodiment of the present invention the enzyme protein content of the enzyme particles is more than 1% w/w, but less than 50% w/w. In another embodiment, the enzyme protein content of the enzyme particles is more than 2% w/w, but less than 25% w/w. In a particular embodiment, the enzyme protein content of the enzyme particles is more than 4% w/w, but less than 20% w/w.

### Organic Filter Aid

Filter aids is a group of substantially inert materials that can be used in filtration pretreatment. An objective of adding filter aids is to improve the flow rate by decreasing cake compressibility and increasing cake permeability.

An organic filter aid, according to the invention, may be a cellulosic or lignocellulosic material. Preferably, the organic filter aid is substantially insoluble in both water and oil at standard ambient conditions (20° C.). Thus, the organic filter aid may be an insoluble cellulose derivative.

In an embodiment, the organic filter aid is a wooden product (such as saw dust), or chemically derived from wood. Preferably, the organic filter aid is a water-insoluble polysaccharide, which may comprise beta(1→4) glycosidic bonds.

In a particularly preferred embodiment, the organic filter aid is cellulose (such as Filtracel from J. Rettenmaier & Sohne, Germany).

The organic filter aid may be mixed with other materials, as long as the mixture retains the overall properties of the organic filter aid, and can be used as a filter aid in oils/fats.

The organic filter aid may even be functionalized with silica, so that a part of the siliceous material used in the particles of the invention is supplied as an integrated part of the organic filter aid.

The enzyme particles of the invention may comprise the organic filter aid in an amount of 10-80% w/w, preferably 20-60% w/w.

### Siliceous Material

The particles of the invention comprise siliceous material. Siliceous material can be amorphous or crystalline or a mixture thereof, and it can be naturally occurring (clay, talc, diatomaceous earth, sand, quartz, etc.) or synthetic (precipitated, fumed, colloidal, silica gels, etc.) that is typically more purified.

Suitable siliceous materials are, for example, commercially available silicas (e.g. Sipernat 22S, Sipernat 50, Sipernat 50s from Evonik, Germany), but also zeolites, diatomaceous earth and kaolins. In a particular embodiment of the present invention the siliceous material is selected from the group consisting of silica, zeolite and kaolin. The siliceous material may have a silica content of greater than 85% w/w, greater than 90%, greater than 95%, or greater than 98%. The siliceous material may be silica with a mean particle size in the range of 1-100 μm, such as 1-50 μm, wherein the silica has a purity of more than 90%. In another embodiment, the siliceous material is a silica with a mean particle size of 1-50 μm and a purity of more than 95%.

The enzyme particles of the invention may comprise the siliceous material in an amount of 10-80% w/w, preferably 20-60% w/w.

### Water-Soluble Polyol

The soluble polyol used in the invention is a carbohydrate or a sugar alcohol, typically with a solubility of at least 0.1 g per 100 ml of water at ambient temperature (e.g. 20° C.). The carbohydrate may consist of 1-20 monosaccharide units. This includes monosaccharides and oligosaccharides such as disaccharides, trisaccharides, maltodextrin and dextrin.

The monosaccharide may be a hexose, either a ketose or an aldose, such as glucose, mannose, galactose, fructose and combinations thereof. Disaccharides may include sucrose, maltose, trehalose, isomaltose, cellubiose, melibiose, primeverose, rutinose, gentiobiose and lactose and combinations thereof. The trisaccharide may be maltotriose, raffinose or a combination thereof.

The carbohydrate may be a starch hydrolysate produced by hydrolysis, e.g. enzymatic hydrolysis, for example with an average of 2-20 monomer glucose units, such as dextrin with DE 6-8 or maltodextrin with DE 20-23 of starch.

The sugar alcohol may be monomeric, e.g. sorbitol or arabitol.

In a particularly preferred embodiment, the polyol is maltodextrin having a DE between 6 and 52. Maltodextrins with a DE above 20 are often referred to as glucose sirup.

The amount of the polyol (carbohydrate or sugar alcohol) used in the particle of the invention may be above 2% by weight, e.g. 2 to 50%, 2 to 30%, 5 to 25% or 7 to 25% by weight of the enzyme particle.

### Uses of the Particles

Particles comprising immobilized lipolytic enzymes, according to the invention, have potential applications in a wide range of enzymatic employed processes such as in the production of pharmaceuticals, specialty commodity chemicals, and vegetable oil processing.

Immobilized enzymes prepared in the context of the invention may be used for hydrolysis, synthesis or modification of organic substances. The hydrolysis, synthesis or modification preferably takes place in a medium essentially devoid of free water.

Accordingly, the invention encompasses a process for enzymatic modification of an organic compound comprising contacting in a reaction medium said organic compound with an immobilized enzyme product according to the invention.

The immobilized enzyme of the present invention may be used for enzymatic modification of an organic compound comprising contacting in a reaction medium said organic compound with an immobilized enzyme produced by the process of the invention.

In a particular embodiment of the present invention the modification is an esterification reaction comprising contacting a first reactant which is a carboxylic acid and a second reactant which is an alcohol with an immobilized lipase of the invention. The carboxylic acid may be selected from but not limited to the group consisting of fatty acids, lactic acid, benzoic acid, acrylic acid and methacrylic acid and the alcohol may be selected from but not limited to the group consisting of methanol, ethanol, isopropanol, polyols such as glycerol, sorbitol, isosorbide, xylitol, glucosides such as ethyl and methyl glucosides, neopentyl alcohol and propylene glycol.

The modification may be a chiral resolvation including an enantioselective synthesis or hydrolysis of carboxylic acid ester or amides; an aldol condensation reaction between two aldehydes; or an epoxidation of olefinic groups by percarboxylic acid produced in situ by the immobilized enzyme.

The modification may be a polyesterification reaction wherein the organic compound to be modified is a hydroxycarboxylic acid or oligomers of such compound e.g. lactic acid or 3-hydroxypropanoic acid. Or the carboxylic acid is a dicarboxylic acid selected from the group consisting of adipic acid, succinic acid, fumaric acid, 2,5-furandicarboxylic acid, glucaric acid, terephthalic acid and isophthalic acid, and the second reactant is selected from the group consisting of polyols such as 1,4-butanediol, 1,6-hexanediol, glycerol, sorbitol, isosorbide, neopentyl alcohol, or propylene glycol.

In another particular embodiment the modification is a ring opening polymerization reaction comprising contacting a lactone with an immobilized lipase produced by the present process. Prepared polymers may be homo or hetero polymers.

The modification may be a transesterification reaction comprising contacting a first reactant which is a carboxylic acid ester and a second reactant which is an alcohol with an immobilized lipase produced by the present process.

The modification may be an interesterification reaction comprising contacting a first reactant which is a carboxylic acid ester and a second reactant which is a second carboxylic acid ester with an immobilized lipase produced by the present process. In a more particular embodiment the modification is an interesterification reaction comprising contacting a first reactant which is a polycarboxylic acid ester and a second reactant which is a second poly-carboxylic acid ester, with an immobilized lipase of the invention.

By interesterification of two different fats/oils the change in fatty acid positions resulting from the interesterification will impact the melting profile of the oils/fat mixture. This is measured by NMR and expressed as the percentage of solid fat at a given temperature in the typical range 10° C.-40° C. Examples of components are coconut fat and palm stearine.

The carboxylic acid ester may be selected from but not limited to the group consisting of alkyl esters of fatty acids, lactic acid, glucaric acid, benzoic acid, acrylic acid, methacrylic acid and wherein the alkyl may be methyl, ethyl, butyl and the alcohol may be selected from the group consisting of but not limited to methanol, ethanol, isopropanol, polyols such as glycerol, alkyl glucosides, such as ethyl glucoside or methyl glucoside, sorbitol, silicone and silicone derivatives, isosorbide, neopentyl alcohol and propylene glycol.

The modification may be a hydrolysis or synthesis producing an enantiopure compound; an amidation reaction comprising contacting a first reactant which is a carboxylic acid and a second reactant which is an amine with an immobilized lipase of the invention.

In a particular embodiment, the modification is an epoxidation reaction comprising in situ production of epoxidation agent with an immobilized enzyme produced by the present process.

In an embodiment of the present invention an immobilized lipase enzyme is used for an esterification, transesterification or interesterification process in a medium essentially devoid of free water. The transesterification may be used for fatty acid substitution, comprising contacting a first reactant and a second reactant with said immobilized lipase by which a substitution reaction occurs.

The first reactant may be a fatty acid ester, preferably a triglyceride or a mixture of triglycerides.

The second reactant may be another fatty acid ester different from the first reactant, preferably a triglyceride or a mixture of triglycerides. Further the second reactant may be an alcohol or a free fatty acid.

The medium in this preferred embodiment of the invention comprises an organic solvent, or it may consist essentially of triglycerides.

Said use of the invention may be applied in production of food products e.g. margarine or cocoa-butter substitutes, for production of esters for e.g. cosmetics, biofuel, etc.

### Processes

The invention also provides a process for conducting a reaction catalyzed by the lipolytic enzyme particles of the invention, comprising:
a) preparing a reaction mixture comprising reactants for the reaction, and
b) contacting the reaction mixture with the immobilized lipolytic enzyme particles at conditions which are effective for conducting the reaction.

The contact may be done by passing the reaction mixture through a packed-bed column of the immobilized lipolytic enzyme, a continuously stirred tank reactor holding the immobilized lipolytic enzyme, a moving bed reactor where the movement of the packed bed of immobilized enzyme is either co-current or counter-current to the reaction mixture, in a batch reactor, optionally with stirring or in any other type of reactor or combination of reactor in which the desired reaction can be carried out.

The lipolytic enzyme may be a lipase, the reactants may comprise a fatty acyl donor and an alcohol, and the reaction may form a fatty acid alkyl ester.

The lipolytic enzyme may be a lipase, the reactants may comprise at least two triglycerides, and the reaction may form different triglycerides. Thus, the reaction may be carried out for a time sufficient to change the melting properties of the mixture of triglycerides.

When the reaction catalyzed by the enzyme particles of the invention is carried out in a (stirred) tank reactor, the enzyme particles may subsequently be separated or recovered from the reactants by way of filtration. After separation, the enzyme particles may be used again (recycled) in the process.

It has been found that a method to re-use the enzyme can be established by allowing the reaction to take place with the enzyme fixed in a filter cake in a filter system. The oil (triglycerides) can be passing the filter cake one or more times to achieve the desired degree of reaction. By operating the reaction in a filter system, it is possible to use already existing equipment and to increase the rate of reaction. The higher rate of reaction is achieved by adding a higher amount of enzyme particles to the filter and enable reuse of the enzyme particles. The deactivation of the enzyme particles, which happens over time, is compensated by adding a small amount of extra enzyme particles to the filter for every batch. This can be repeated until the maximum filter cake thickness is reached, and the filter is full and/or the maximum pressure drop over the filter is reached.

### The Present Invention is Further Described by the Following Numbered Embodiments:

Embodiment 1. (a plurality of) Enzyme particles comprising a lipolytic enzyme, a siliceous material, an organic filter aid, and a water-soluble polyol selected from carbohydrates and sugar alcohols.

Embodiment 2. The particles of embodiment 1, which comprise the siliceous material in an amount of 10-80% w/w.

Embodiment 3. The particles of embodiment 1 or 2, which comprise the siliceous material in an amount of 20-60% w/w.

Embodiment 4. The particles of any of embodiments 1-3, which comprise the organic filter aid in an amount of 10-80% w/w.

Embodiment 5. The particles of any of embodiments 1-4, which comprise the organic filter aid in an amount of 20-60% w/w.

Embodiment 6. The particles of any of embodiments 1-5, which comprise the siliceous material and the organic filter aid in a total amount of 40-95% w/w

Embodiment 7. The particles of any of embodiments 1-6, which comprise the siliceous material and the organic filter aid in a total amount of 50-90% w/w

Embodiment 8. The particles of any of embodiments 1-7, which comprise the polyol in an amount of 2-50% w/w.

Embodiment 9. The particles of any of embodiments 1-8, which comprise the polyol in an amount of 5-25% w/w.

Embodiment 10. The particles of any of embodiments 1-9, which comprise the lipolytic enzyme in an amount of 1-50% w/w.

Embodiment 11. The particles of any of embodiments 1-10, which comprise the lipolytic enzyme in an amount of 2-25% w/w.

Embodiment 12. The particles of any of embodiments 1-11, which comprise the lipolytic enzyme in an amount of 4-20% w/w.

Embodiment 13. The particles of any of embodiments 1-12, wherein the siliceous material is silica, kaolin, diatomaceous earth or zeolite.

Embodiment 14. The particles of any of embodiments 1-13, wherein the siliceous material is silica.

Embodiment 15. The particles of any of embodiments 1-14, wherein the siliceous material is fumed silica.

Embodiment 16. The particles of any of embodiments 1-15, wherein the organic filter aid is a water-insoluble polysaccharide.

Embodiment 17. The particles of any of embodiments 1-16, wherein the organic filter aid is a water-insoluble polysaccharide comprising beta(1-4) glycosidic bonds.

Embodiment 18. The particles of any of embodiments 1-17, wherein the organic filter aid is a cellulosic or lignocellulosic material.

Embodiment 19. The particles of any of embodiments 1-18, wherein the organic filter aid is derived from wood.

Embodiment 20. The particles of any of embodiments 1-19, wherein the organic filter aid is cellulose.

Embodiment 21. The particles of any of embodiments 1-20, wherein the polyol is selected from the group consisting of dextrin, maltodextrin, trisaccharides, disaccharides, monosaccharides, and mixtures thereof.

Embodiment 22. The particles of any of embodiments 1-21, wherein the polyol is selected from the group consisting of sucrose, maltose, trehalose, isomaltose, cellubiose, melibiose, primeverose, rutinose, gentiobiose, lactose, and mixtures thereof.

Embodiment 23. The particles of any of embodiments 1-22, wherein the polyol is selected from the group consisting of glucose, mannose, galactose, fructose, and mixtures thereof.

Embodiment 24. The particles of any of embodiments 1-23, wherein the polyol is maltodextrin having a DE between 6 and 52.

Embodiment 25. The particles of any of embodiments 1-24, wherein the lipolytic enzyme is a lipase, cutinase or phospholipase.

Embodiment 26. The particles of any of embodiments 1-25, wherein the lipolytic enzyme is a lipase.

Embodiment 27. The particles of any of embodiments 1-26, which further comprise an alkaline buffer component.

Embodiment 28. The particles of any of embodiments 1-27, which further comprise a carbonate.

Embodiment 29. The particles of any of embodiments 1-28, which further comprise sodium carbonate or potassium carbonate.

Embodiment 30. The particles of any of embodiments 1-29, which is a substantially homogenous composition of the ingredients.

Embodiment 31. The particles of any of embodiments 1-30, which are prepared by spray drying, or another drying technique.

Embodiment 32. The particles of any of embodiments 1-31, which are prepared by absorption of the enzyme and/or the polyol into a mixture of the siliceous material and organic filter aid.

Embodiment 33. The particles of any of embodiments 1-32, which are comprised in a pellet prepared by compression.

Embodiment 34. The particles of any of embodiments 1-33, which are comprised in an extrudate.

Embodiment 35. The particles of any of embodiments 1-34, which are comprised in a block of fat.

Embodiment 36. The particles of any of embodiments 1-35, which have a particle size below 100 μm.

Embodiment 37. The particles of any of embodiments 1-36, which have a particle size of 1-60 μm.

Embodiment 38. The particles of any of embodiments 1-37, which have a particle size of 2-40 μm.

Embodiment 39. The particles of any of embodiments 1-38, which have a particle size of 5-30 μm.

Embodiment 40. The particles of any of embodiments 1-39, which further comprise a coating.

Embodiment 41. The particles of any of embodiments 1-40, which further comprise a triglyceride coating.

Embodiment 42. The particles of any of embodiments 1-41, which are encapsulated in oil.

Embodiment 43. The particles of any of embodiments 1-42, which are encapsulated in a plant derived oil.

Embodiment 44. The particles of any of embodiments 1-43, which have a water content of less than 40% w/w.

Embodiment 45. The particles of any of embodiments 1-44, which have a water content of less than 25% w/w.

Embodiment 46. The particles of any of embodiments 1-45, which have a water content of less than 10′)/0 w/w.

Embodiment 47. The particles of any of embodiments 1-46, which have a water content of less than 5% w/w.

Embodiment 48. The particles of any of embodiments 1-47, which are encapsulated in oil or fat.

Embodiment 49. A method for enzymatic interesterification, comprising contacting a mixture of triglycerides with the particles of any of embodiments 1-48.

Embodiment 50. The method of embodiment 49, wherein the triglycerides are contacted with the particles for a time sufficient to change the melting properties of the mixture of triglycerides.

Embodiment 51. The method of embodiment 49 or 50, wherein the mixture of triglycerides is contacted with the particles in a stirred-tank reactor.

Embodiment 52. The method of any of embodiments 49-51, wherein the mixture of triglycerides is subsequently separated from the particles in a filtration step.

Embodiment 53. The method of embodiment 49 or 50, which is carried out in a filter bed containing the particles.

Embodiment 54. The method of any of embodiments 49-53, wherein the particles are recovered and used again in a method according to any of embodiments 49-53.

Embodiment 55. A powder or slurry/suspension comprising the particles of any of embodiments 1-48 and at least 10% oil or fat.

Embodiment 56. A powder or slurry/suspension comprising the particles of any of embodiments 1-48 and at least 10% plant derived oil or fat.

The present invention is further described by the following examples which should not be construed as limiting the scope of the invention.

### EXAMPLES

Chemicals were commercial products of at least reagent grade.

### Example 1

### Analysis of the SFC. Describing the Method of Analysis

The properties of enzymatic interesterified fat (EIE) is measured by determining the percentage of solid fat content at one or more temperatures according to the AOCS Official Method Cd 16b-93 “Solid Fat Content (SFC) by Low-Resolution Nuclear Magnetic Resonance”.

With this method, the SFC is defined as the ratio, expressed as a percentage between the NMR response obtained from the hydrogen nuclei in the solid phase of the sample and the NMR response obtained from the hydrogen nuclei in both the solid and the liquid phase of the sample.

The fat sample is temperature adjusted to the given temperature. Suitable temperatures are 10° C., 15° C., 20° C., 25° C., 30° C., 35° C., 40° C., 45° C., 50° C., 55° C., or 60° C. as required.

### Example 2

### Comparison Filtracel to Filtracel+Silica

In this experiment, we tested the impact of carrier composition in the efficiency of interesterification (lower SFC). Two products were tested:

Sample 4B was made by spraying enzyme concentrate (6.3 g per 100 g carrier) on a carrier consisting of only Filtracel ESG 950 from J. Rettenmaier & Sohne, Germany. In addition, a solution of maltodextrin MD-20 was added before the product was dried. Drying in a heating cabinet overnight.

Sample 16B was made similar to sample 4B, but by adding the double amount of enzyme and 30% more maltodextrin and using a carrier consisting of 50% Filtracel ESG 950 and 50% Sipernat 25 (Silica from Evonik, Germany).

The dried enzyme samples were incubated with a fat blend of 70% palm stearine +30% coconut oil at 80° C. for 4-5 hours. The dosage and the SFC result after reaction is shown in Table 1 below. The SFC at 40° C. for the starting mixture is 15%.

TABLE 1
Filtracel vs Filtracel/Sipernat.

| | | Product | Reaction | SFC at |
|---|---|---|---|---|
| Product | Carrier | dosage | time | 40° C. |
| 4B | 100% Filtracel | 4.0% | 4 hours | 7.1% |
| 4B | 100% Filtracel | 1.0% | 5 hours | 12.6% |
| 16B | 50/50% Filtracel | 0.5% | 5 hours | 4.5% |
| | & Sipernat | | | |

It can be seen from the SFC data in Table 1 that the 16B sample is by far the most efficient in achieving a low SFC %.

### Example 3

### Effect of Maltodextrin on Enzyme Performance

In this experiment, we evaluated the effect of the maltodextrin addition to the enzyme efficiency. The enzyme product was made from spraying enzyme concentrate and maltodextrin on a carrier consisting of 1:1 Sibernat 25 and Filtracel ESG 950.

To 100 g carrier was added 66 g enzyme concentrate (corresponds to 12.6 g dry matter), and the amount of maltodextrin as shown in Table 2. The maltodextrin was added dissolved in 34 g water. The material was dried and used for the interesterification experiment at 80° C. for up to 5 hours. Dosage of the enzyme products was 0.2% for all three.

TABLE 2
Effect of maltodextrin on SFC

| | Mean SFC | Mean SFC |
|---|---|---|
| | at 40° C. | at 40° C. |
| Maltodextrin | (2 hours | (5 hours |
| | reaction) | reaction) |
| 0% | 13.3% | 12.6% |
| 4% | 10.3% | 8.2% |
| 8% | 9.8% | 7.7% |

From the data in Table 2, it can be seen that addition of 8% maltodextrin is superior to 4%, which is much better than no maltodextrin addition.

### Example 4

### Compare Small Silica to TL IM

In this experiment, sample 16B from Example 2 was compared to the Lipozyme TL IM product from Novozymes, Denmark. The TL IM product is the industry standard used today in a column technology and continuous production for EIE. The experiments were made in batch operation in the same way as described in Example 2.

TABLE 3
Filtracel/Sipernat vs TL IM.

| | | Product | Reaction | SFC at |
|---|---|---|---|---|
| Product | Carrier | dosage | time | 40° C. |
| TL IM | Large silica | 4.0% | 5 hours | 3.8% |
| | carrier | | | |
| 16B | 50/50% Filtracel | 0.5% | 5 hours | 4.5% |
| | & Sipernat | | | |

The SFC for the two samples are similar even the dosage of enzyme product for TL IM is 4% and only 0.5% for sample 16B. This shows the efficiency of reaction is far better for sample 16B.

1. A plurality of enzyme particles, wherein said particles comprise a lipolytic enzyme, a siliceous material, an organic filter aid, and a water-soluble polyol selected from carbohydrates and sugar alcohols.

2. The particles of claim 1, which comprise the siliceous material in an amount of 10-80% w/w.

3. The particles of claim 1, which comprise the organic filter aid in an amount of 10-80% w/w.

4. The particles of claim 1, which comprise the siliceous material and the organic filter aid in a total amount of 40-95% w/w.

5. The particles of claim 1, which comprise the polyol in an amount of 2-50% w/w.

6. The particles of claim 1, which comprise the lipolytic enzyme in an amount of 1-50% w/w.

7. The particles of claim 1, wherein the siliceous material is silica.

8. The particles of claim 1, wherein the organic filter aid is a water-insoluble polysaccharide.

9. The particles of claim 1, wherein the organic filter aid is cellulose.

10. The particles of claim 1, wherein the polyol is selected from the group consisting of dextrin, maltodextrin, trisaccharides, disaccharides, monosaccharides, and mixtures thereof.

11. The particles of claim 1, wherein the polyol is maltodextrin having a DE between 6 and 52.

12. The particles of claim 1, wherein the lipolytic enzyme is a lipase.

13. The particles of claim 1, which is a substantially homogenous composition of the ingredients, prepared by spray drying or absorption, followed by drying.

14. The particles of claim 1, which has an average diameter below 100 μm.

15. A powder or a slurry/suspension comprising the particles of claim 1 and at least 10% oil or fat.

16. A method for enzymatic interesterification, comprising contacting a mixture of triglycerides with the particles according to claim 1.

17. The method of claim 16, wherein the mixture of triglycerides is contacted with the particles in a stirred-tank reactor, and the mixture of triglycerides is subsequently separated from the particles in a filtration step.

18. The method of claim 16, wherein the mixture of triglycerides is contacted with the particles in a filter system by one or more passes of the mixture of triglycerides through a filter system with a filter cake comprising or consisting of the enzyme particles.
